(19) United States
(12) Patent Application Publication
(10) Pub. No.: US 2020/0315754 A1
(43) Pub. Date: Oct. 8, 2020

(54) AUTOMATED DENTAL TREATMENT SYSTEM

(71) Applicant: Cyberdontics Inc., Vancouver (CA)

(72) Inventors: Christopher John CIRIELLO, Castlegar (CA); Christine Eunkyung PARK, Victoria (CA); Nathan John MULLER, Victoria (CA); Dustin Richard DEMONTIGNY, Victoria (CA)

(21) Appl. No.: 16/486,662

(22) PCT Filed: Feb. 22, 2018
(86) PCT No.: PCT/IB2018/051115

§ 371 Date: Aug. 16, 2019

Related U.S. Application Data
(60) Provisional application No. 62/461,896, filed on Feb. 22, 2017.
(60) Provisional application No. 62/515,106, filed on Jun. 5, 2017.
(60) Provisional application No. 62/532,641, filed on Jul. 14, 2017.
(60) Provisional application No. 62/609,550, filed on Dec. 22, 2017.

(51) Int. Cl. A61C 13/00; A61B 34/10; A61B 34/20; A61C 3/02

(52) U.S. Cl. CPC A61C 13/0004; A61B 34/10; A61B 34/20; A61B 2034/2057; A61C 13/0019; A61B 2034/104; A61C 2201/005; A61C 3/02

(57) ABSTRACT

A system for performing dental surgery on a subject includes a central processing unit that controls automated operation of the system and a display that renders an image of a target tooth requiring surgical intervention. The image of a target tooth is created from an image file received from the CPU. An input device receives surgical instructions from a user for providing the surgical intervention with the instructions being subsequently received by the central processing unit. The instructions include visual indications on the image of a target tooth that are to be treated. The system also includes a segmented dental handpiece that has a first segment attached to a second segment. The second segment is attached to a dental drill head and is movable with respect to the first segment under control of the CPU. The first segment can be held stationary relative to the target tooth by an operator.

### CROSS-REFERENCE TO RELATED APPLICATIONS

This application is filed pursuant to 35 U.S.C. § 371 as a United States National Phase Application of International Application No. PCT/IB2018/051115, filed Feb. 22, 2018, which claims the benefit of U.S. provisional application Ser. No. 62/461,896 filed Feb. 22, 2017; 62/515,106 filed Jun. 5, 2017; 62/532,641 filed Jul. 14, 2017; and 62/609,550 filed Dec. 22, 2017, the disclosures of which are hereby incorporated in their entirety by reference herein.

### TECHNICAL FIELD

In at least one aspect, the present invention is related to automated controlled systems for treating dental disease.

### BACKGROUND

Although advances have been made in recent years for the treatment of specific dental diseases, the actual delivery of dental treatment remains a manually intensive process. Accordingly, there is a need for methodology for automating dental treatment.

### SUMMARY

The present invention solves one or more problems of the prior art by providing in at least one embodiment, a system for performing dental surgery on a subject. The system includes a central processing unit that controls automated operation of the system and a display that renders an image of a target tooth requiring surgical intervention. The image of a target tooth is created from an image file received from the central processing unit. An input device receives surgical instructions from a user for providing the surgical intervention with the surgical instructions being subsequently received by the central processing unit. The surgical instructions include visual indications on the image of a target tooth that is to be treated. The system also includes a segmented dental handpiece that has a first segment attached to a second segment. The second segment is attached to a dental drill head and is movable with respect to the first segment under control of the central processing unit. The first segment can be held stationary relative to the target tooth by an operator. Characteristically, the dental drill head includes a dental burr protruding therefrom. The system can also include a three-dimensional (3D) vision system that includes a plurality of cameras attached to the segmented dental handpiece. The plurality of cameras provides two dimensional images and/or live video of a subject's teeth to be mapped to a predetermined 3D surface scan of a surgical site thereby establishing a world coordinate system to which the segmented dental handpiece is registered.

In another embodiment, a method for pre-milling a dental crown is provided. The method includes a step of providing the system for performing dental surgery set forth herein. The dental crown is pre-milled prior to a surgical appointment wherein CAD/CAM milling technology is combined with the segmented dental handpiece (i.e., a smart drill) and dental surgery is pre-planned between the diagnostic and surgical appointment based on custom 3D mesh data merged with other diagnostic modalities since the system has programmed therein the shape it is cutting into the tooth before it cuts it. The segmented dental handpiece can execute the pre-planned surgery with sub millimeter precision cutting the desired shape into the tooth with the milling system milling a crown insert with a mating tooth to crown interface surface before the surgery appointment thereby allowing the crown to be ready chairside at the surgery appointment such that once the surgery is complete the crown is immediately inserted.

Advantageously, the segmented drill can be used in oral surgery, endodontics (root canals), operative dentistry (fillings), and dental, Implant placement and in general any use a dental drill is currently used for but not listed here.

### BRIEF DESCRIPTION OF THE DRAWINGS

FIG. 1A is a schematic illustration of a dental treatment system;

FIG. 1B is an idealized depiction showing the interaction between the components of the dental treatment system;

FIG. 2A is a schematic cross section of a segmented dental handpiece;

FIG. 2B is a schematic of an alternate design for the segmented dental handpiece;

FIG. 3A is a cross section of a translation shaft section used in the segmented dental handpiece;

FIG. 3B is a longitudinal section of a translation shaft section used in the segmented dental handpiece;

FIG. 4 is a perspective view showing a drilling head with water jets emerging therefrom;

FIGS. 5A and 5B depict the movement achieved by the segmented dental handpiece;

FIG. 6 provides a pictogram of all teeth in the upper and lower arch of a subject;

FIG. 7 illustrates a user interface displaying a radiograph registered to a 3D mesh representation of the tooth acquired from a 3d scanning system; and

FIG. 8 displays a user interface for parametrically generating a tool path from 3 user clicks—click 1—the location of the external surface of the tooth to be prepared, click 2—the preparation margin width, click 3 captures both the taper and the occlusal reduction. The other parameters are determined when the user selects the tooth to be prepared and the restoration material.

FIG. 9 illustrates the pre-milling of dental crowns.

FIGS. 10-13 provide schematics of an augmented reality system that can be use with the segmented drill system of the present invention.

### DETAILED DESCRIPTION

As required, detailed embodiments of the present invention are disclosed herein; however, it is to be understood that the disclosed embodiments are merely exemplary of the invention that may be embodied in various and alternative forms. The figures are not necessarily to scale; some features may be exaggerated or minimized to show details of components. Therefore, specific structural and functional details disclosed herein are not to be interpreted as limiting, but merely as a representative basis for teaching one skilled in the art to variously employ the present invention.

The term “subject” as used herein refers to a human patient in need of dental treatment.

The term “STL” is a file format for stereolithographic CAD software.

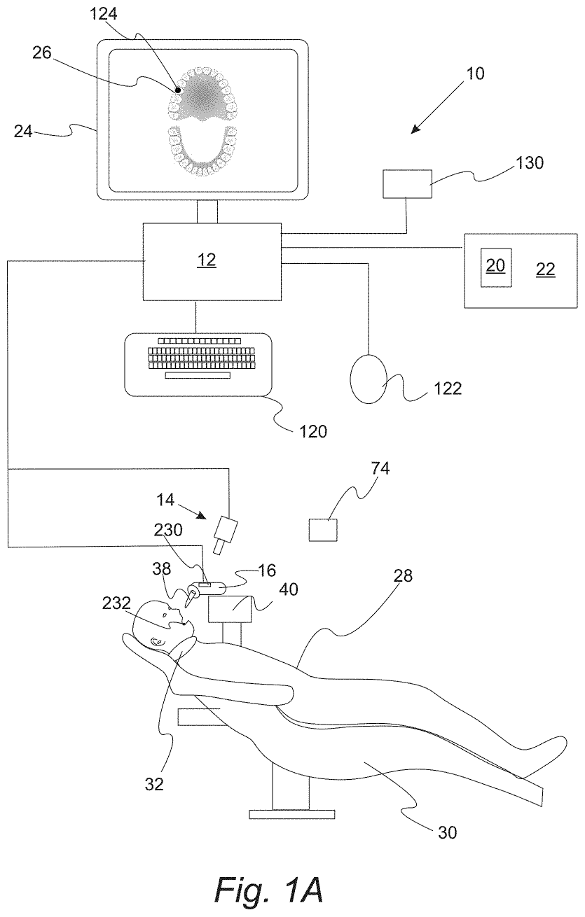

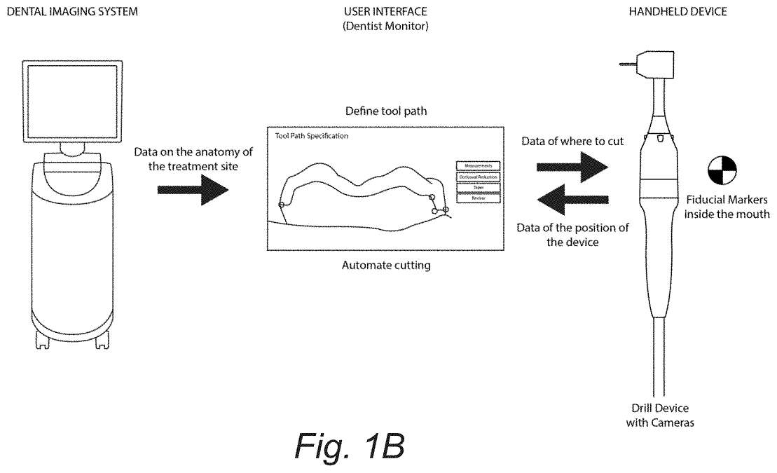

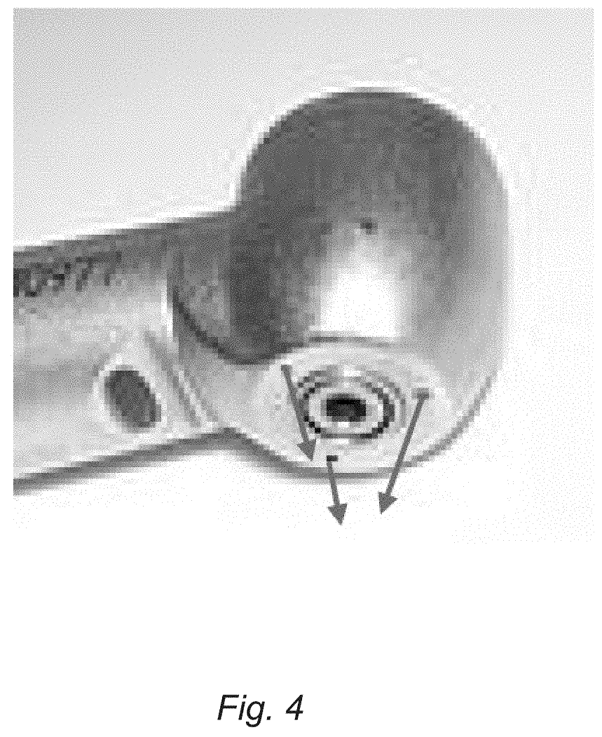

With reference to FIG. 1, a schematic illustration of an automated dental treatment system is provided. Dental treatment system 10 includes central processing unit 12 in communication with tooth scanner 14 and segmented dental handpiece 16. Segmented dental handpiece 16 is held by a user (i.e., a dentist) and possesses a number of robotic movement features. In a refinement, tooth scanner 14 includes user handle for a user to hold and move the scanner as needed. Central processing unit 12 controls automated operation of the dental treatment system 10 and can monitor tooth cutting performance. The central processing unit can also provide feedback from a burr that is used to monitor burr contact indicating unplanned cutting or to detect tooth decay. Typically, central processing unit 12 is contained in a computer work station. Control program(s) 20 which resides in computer memory 22 is executed by central processing unit 12 to receive image files from scanner 14 and to at least partially control the movement of segmented dental handpiece 16. During operation, tooth scanner 14 can transfer tooth image data to the central processing unit 12. The central processing unit 12 includes a display 24 on which the surgical process is guided through a series of onscreen prompts. Display 24 renders an image 26 of a target tooth in subject 28 from an image file requiring surgical intervention. FIG. 1 depicts subject 28 sitting in dental chair 30. Subject 28's head can be immobilized by head restraint 32. Segmented dental handpiece 16 includes an end effector 38 extending therefrom for performing dental surgery. End effector 38 is typically a dental burr. In another refinement, dental treatment system 10 includes passive positioning encoder arm 40 which tracks the patient position and relays it to central processing unit 12. In a variation as depicted in FIG. 4, system 10 includes a cooling water jet that is used for position tracking, the water cooling jet providing an ultrasound signal or a light signal along a fiber optic axially down the cooling water jet to calculate distance to the target tooth.

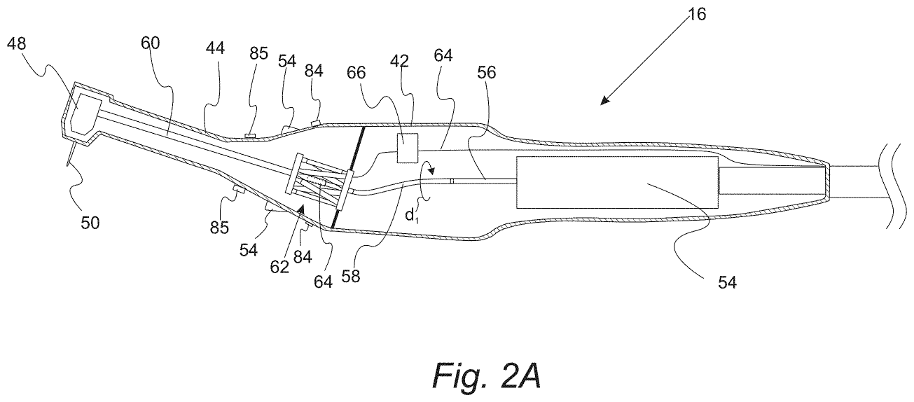

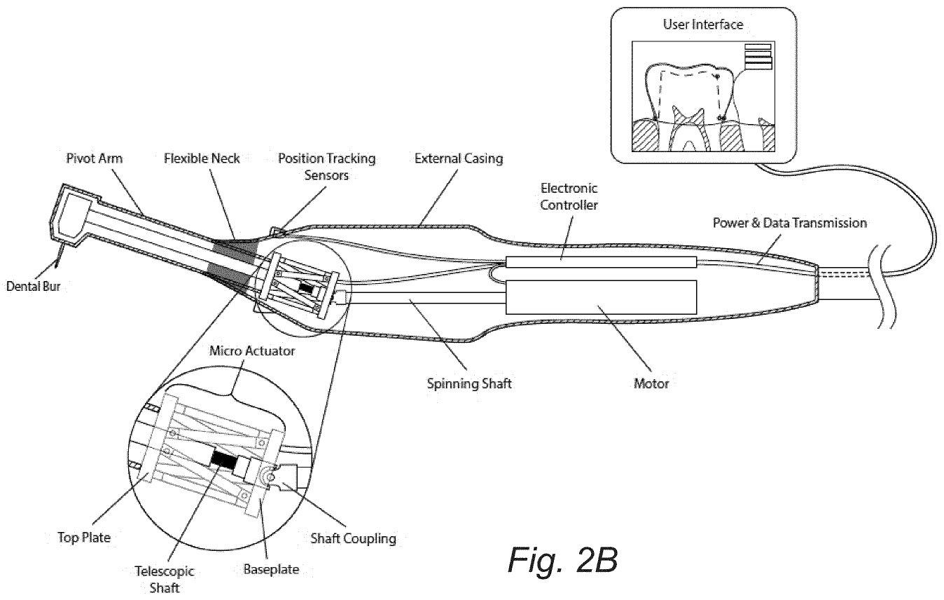

With reference to FIG. 2A, a schematic cross section of the segmented dental handpiece is provided. Segmented dental handpiece 16 includes first segment 42 attached to a second segment 44 via flexible neck 46. Second segment attached 44 to a standard dental drill head 48 which includes a dental burr 50. Second segment 44 is attached to a standard dental drill head 48 which includes a dental burr 50. Second segment 44 is moveable with respect to the first segment 42 under control of the central processing unit 12. Typically, first segment 42 is held stationary relative to the target tooth by an operator. The variation depicted in FIG. 2 includes motor 54 that rotates drive shaft 56 along direction d₁. Shaft 56 is coupled to flexible shaft 58 which is also coupled to shaft 60 which drives dental drill head 48.

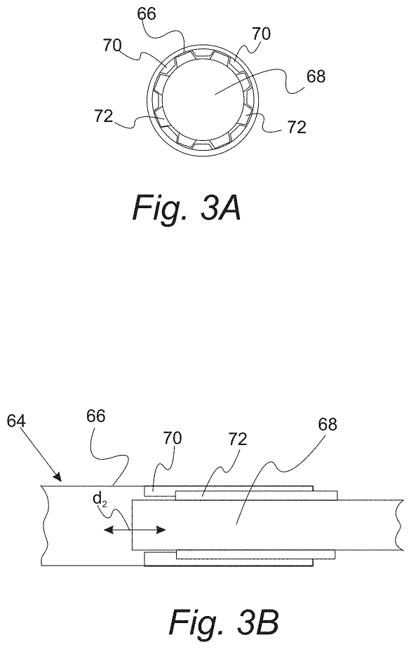

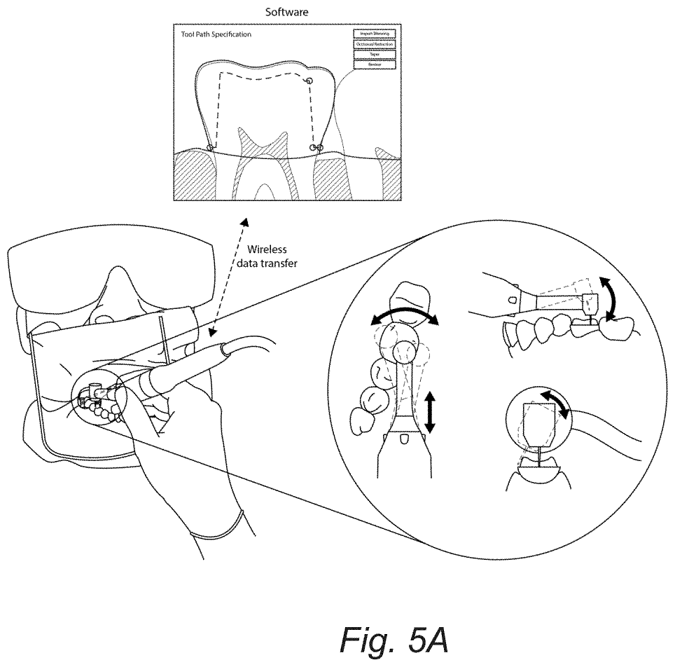

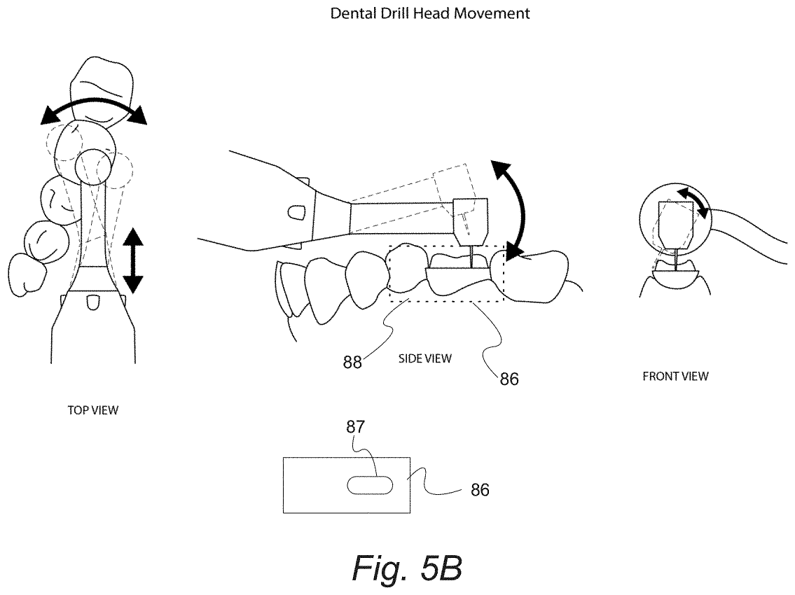

Micro actuation system 62 moves the second segment 44 relative to the first segment by moving shaft 60. Advantageously, the second segment is movable by micro actuation system 62 relative to the first segment with 3 to 6 degrees of freedom. In particular, second segment 44 is moveable with 6 degrees of freedom (i.e., 3 translational and 3 angular degrees of freedom). FIGS. 5A and 5B depict the movement achieved by the segmented dental handpiece while following a tooth path to cut a targeted tooth. In a refinement, micro actuation system 62 includes a hexapod micro actuator. Micro actuation system is controlled by the central processing unit 12 via control line 64 with optional actuation control board 66. In order to properly move second segment 44, micro actuation system 62 is mounted to or is fixed relative to first segment 42. Moreover, shaft 60 includes a translational section 64 that includes first rod section 66 that is moveable with respect to second rod section 68 along direction d₂. FIG. 3A is a cross section of translational section 64 while FIG. 3B is a longitudinal section of translational section 64. First rod section 66 includes teeth 70 that interlock with teeth 72 extending from second rod section 68. Translational section 64 along with flexible shaft 58 allows second segment 44 to achieve the six degrees of freedom. In a refinement, micro actuation system 62 can include 1, 2, 3 or more piezoelectric elements that moves the second segment 44 relative to the first segment. In another refinement, micro actuation system 62 can be operated in a mode with less than 6 degrees of freedom (e.g., 1, 2, 3, or 4) with a user holding the drill providing the addition degrees of freedom as needed.

With reference to FIG. 2B, an alternate design for segmented dental handpiece 16 is provided. In this variation, the system is brought into the field of operation and held by an operator. Initial Position data is recorded by the Position Tracking Sensors and sent to the User Interface. This determines the device's location within the mouth by reading a datum rigidly coupled to the teeth. With a set location, a toolpath is generated and Data is sent back to the Electronic Controller and read out to the operator via a User Interface. The operator then generates a toolpath based on interactions with the User Interface, which is then transferred back to the Electronic Controller and into the Motor and Micro Actuator. This executes a cutting operation by setting both rotary speed of the Dental Bur via the Motor and the position of the Dental Bur via the Micro Actuator. The rotary speed is transferred from the Motor through the Spinning Shaft, which is coupled at an angle through a Shaft Coupling. This is then propagated up the pivot arm through a Telescopic Shaft joint and an extension of Spinning Shaft to the Dental Bur. The Dental Bur is positioned through the Pivot Arm with the Micro Actuator in 4 degrees of freedom. In order to enable this: the pivot arm is rigidly fixed to the Top Plate of the Micro Actuator; the Baseplate is rigidly fixed to the External Casing; the Telescopic Shaft allows for axial displacement; the Flexible Neck allows for movement in all axes.

In a variation, the segmented drill manipulated by a user to make broad cuts while the segmented drill automatically makes finer cuts.

With reference to FIG. 4, standard dental drills are equipped with multiple water nozzles that spray water onto the treatment area while cutting to prevent overheating and killing the nerves in the tooth. In a refinement, a beam of ultrasound or fiber optic light axially is down the jet and use information about the distance to the irrigated object (treatment tooth) to provide additional position information.

In a variation, the three-dimensional vision system includes an internal vision system. For example, a plurality of cameras 54 attached to the segmented dental handpiece 16. The plurality of cameras 54 provide two dimensional images and/or live video of a subject's teeth to be mapped to a predetermined 3D surface scan of a surgical site thereby establishing a world coordinate system to which the segmented dental handpiece is registered. In one refinement, the plurality of cameras 54 includes millimeter scale cameras. Advantageously, the vision system can be adapted to use fiducial markers to locate itself in the world coordinate system. Examples of fiducial markers include, but are not limited to, neighboring teeth, optical markers attached to teeth, or dental claps or similar devices. In a refinement, the fiducial markers include active emitters to enhance or improve ability of the plurality of cameras to detect or monitor the fiducial markers. The predetermined 3d surface scan can be generated by an intra oral 3d surface scanner (e.g., tooth scanner 14). In a refinement, the vision system supports six degrees of freedom. Advantageously, a boundary for maneuvers of the second segment 44 is defined at least in part by the vision system.

As set forth below in more detail, output of the vision system is presented in a three-dimensional format either as an overlay onto an imaged anatomy or as a secondary image. In particular, central processing unit 12 parametrically generates a 3D mesh of a post-surgical site, and a surgical tool path to be cut into the target tooth. Central processing unit 12 can execute an algorithm for registering an anatomical image (e.g., a radiograph) onto a 3d mesh of the surgical site. Moreover, the anatomical image, a registered anatomical image, a 3d representation of the surgical site, and a 3d representation of the target tooth as it will appear post surgically are presented on the display to aid the operator in altering or approving a toolpath generated prior to initiating surgery.

In some variations, the system provides operator feedback that guides the operator in positioning of the segmented dental handpiece correctly over the surgical site. Examples of operator feedback include tactile or auditory feedback. In some variations, a surgical operation is unlocked (i.e., it is permitted) once the segmented hand piece 16 is in a correct position.

In still another variation, an external vision system in which camera(s) are external to the segmented dental handpiece 16. In FIG. 1, this external vision system 74 visualize the segmented dental handpiece and track its motion.

In yet another variation, the segmented dental handpiece 16 can be used without a vison system. Instead, the segmented dental handpiece 16 can be directly fixed to the tooth (removing the requirement for a vision system). This is an alternative to being held by the dentist and motion stabilized with vision system.

In still another variation as depicted in FIG. 2A, segmented dental handpiece 16 active emitters 84 for the fiducials in the vision system. The fiducials emit light or energy/magnetic waves and the detector 85 in the drill would coordinate those multiple fiducials to determine location. This can account for the debris that is flying in the patients mouth than may interfere or obstruct a traditional optical vision system.

As depicted in FIG. 5B, guide splits 86 can be used to hold segmented dental handpiece 16 in certain orientations to cut teeth to fit crowns. For example, the motion of segmented dental handpiece 16 can be restricted by slot 87 in guide split 86. Side sections 88 hold the guide split over the tooth. With this variation, a crown can be made first. A tooth can be cut with the segmented dental handpiece 16 to fit the crown.

With reference to FIGS. 1-8, operation of dental treatment 10 is described as follows. Central processing unit 12 controls segmented dental handpiece 16 to remove a region of the target tooth. Dental treatment system 10 includes input devices 120, 122 which can for example be a keyboard and mouse that receive surgical instructions from a user for providing the surgical intervention. The instructions are received by the central processing unit 12. Characteristically, the surgical instructions including visual indications 124 on the image of a target tooth that are to be treated. Control program 20 guides the user through the dental protocols through a series of onscreen prompts (i.e., the user interface). In this context, actions attributable to control program 20 is understood to mean the execution of the relevant steps by central processing unit 12. In a variation, dental treatment system 10 includes static memory 130 for storing patient profiles and records which can be accessed by the user. In a refinement, central processing unit 12 also displays a load screen that shows a series of patient records and gives the option to load an existing patient, or create a new patient record.

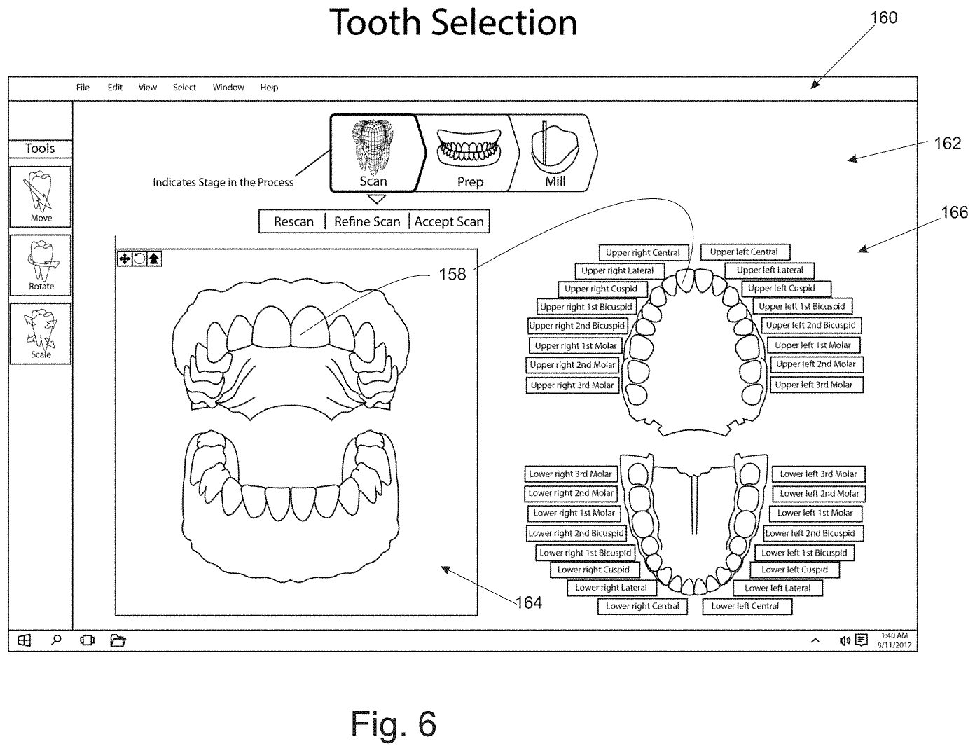

During operation of the dental treatment system, the user creates a patient profile entry. Such a profile includes the patient name, date of birth, patient number, and the like. The user is given the option of entering a treatment plan. In a refinement, the central processing unit renders a pictogram of all teeth in an upper and lower arch of the subject on the display as depicted in FIG. 6. Control program 20 also provides an onscreen prompt that allows the user to enter specific information above the teeth. Typically, this information is entered by clicking on specific teeth on the user interface. For example, missing teeth or the tooth on which surgery is to be performed are identified in this manner with descriptive notes being enterable. Moreover, the user can identify the type of restoration associated with the selected tooth such as crown, bridge, onlay/inlay, and filling (e.g., class 1 to 5). Control program 20 has the capability to make various suggestions to the user. For example, the central processing unit 12 presents to the user on display 24 (through execution of control program 20) a selection for teeth to place a modified rubber dam clamp on for a dental procedure and/or candidate teeth to scan.

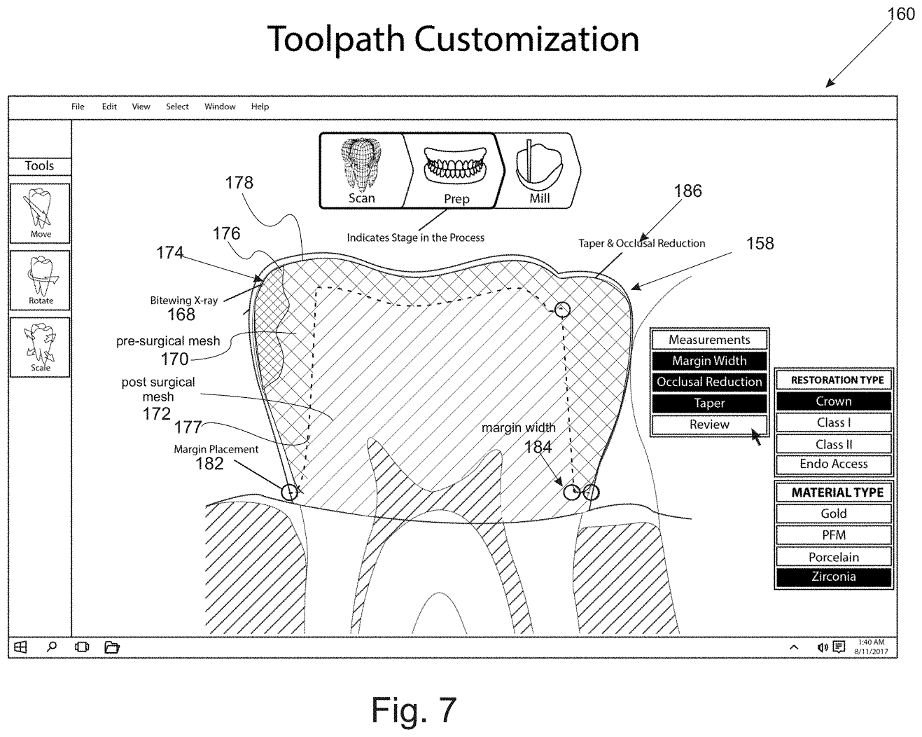

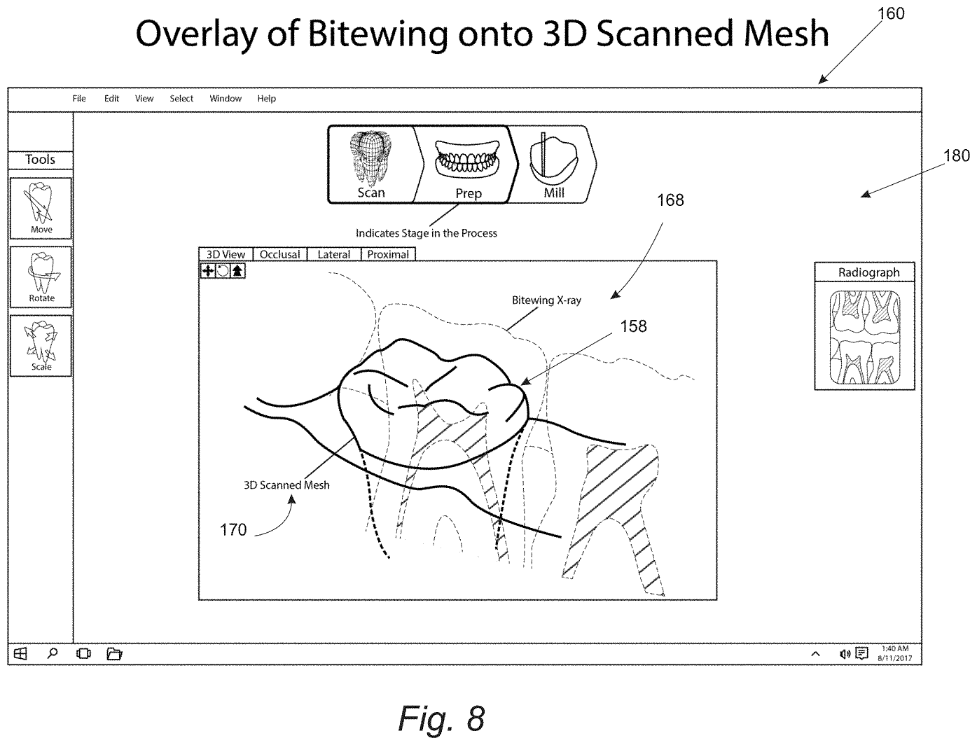

Referring to FIG. 1, automated drill system 10 includes a dental tooth scanner 14 to create an image of the subject's tooth. An example of a dental tooth scanner is the iTero® Element which is an intraoral scanner commercially available from Align Technology, Inc. Such dental tooth scanners create an STL image file of the region of the patient's dental arches that are to be worked on. In particular, control program 20 creates a 3D image from the data received from dental tooth scanner 14. In a variation, central processing unit 12 renders a three-dimensional model of the target tooth using a deformable pre-surgical mesh with a bitewing X-ray overlaid. The three-dimensional model is typically presented on display 24. Advantageously, the deformable pre-surgical mesh is editable by the user to produce a postsurgical mesh and a representation of the target restoration (restoration mesh). The restoration mesh is generated with an inner surface that aligns the surface of the post-surgical mesh and an outer surface that represents the outer surface of the desired tooth restoration. Typically, during this editing, the tooth is not shaped as it is in a broken state, but as it should be in a pristine state which is designated as the completed restoration. The three-dimensional model presents a rendering of surgical criteria to the user regarding treatment. Such surgical criteria include occlusal contacts, proximal contacts, anatomical morphology of the target tooth, occlusal reduction, margin placement relative to biological width and proximal contacts, and an image of a bite-wing superimposed onto the pre-surgical mesh. Occlusal contacts are the precise contact points on the completed restoration tooth anatomy where the upper and lower teeth contact one another. Therefore, the three-dimensional model ensures that the restored teeth are positioned against the opposite teeth correctly. Proximal contacts relate to how the completed restoration touches the teeth on each side of it. Therefore, the three-dimensional model ensures that the restored teeth fit correctly between the adjacent teeth. Finally, with respect to anatomical morphology, the three-dimensional model ensures that the restored teeth look anatomically correct (e.g. cervical and buccal heights of contour). In addition, central processing unit 12 can display clinical information relevant to the surgical criteria. For example, as depicted in FIG. 7, the user interface displays tooth intersection points (labeled with item number 140) on the mesh. This allows the user to reduce or re-contour this area of the mesh to the give the correct contacts with opposing and adjacent teeth or anatomical shape (see, FIG. 7). The tool path is parametrically generated from a view port with a bitewing or periapical radiograph registered to the 3d mesh (see. FIG. 8). This allows the clinical to avoid anatomical landmarks such as the pulp, adjacent teeth, or remove caries. The tool path is then parametrically generated with 6 user inputs: tooth number being operated on, the restoration material which determines the material thickness required, the opposing dental arch mesh, and 3 clicks. The three clicks are (1—margin placement), 2 margin widths, and 3 preparation wall taper and occlusal reduction.

Once parametrically generated tool path is approved by the dentist, it is used for the simultaneous milling of the restoration in the milling chamber concurrently while the segmented dental handpiece and end effector is cutting the tooth. It should be noted that the internal surface and margins of the filling are not milled until the tooth preparation shape is completed to ensure that the filling is made once the final internal structure is known. At this point, the only surface in question would be the mating/internal surface of the tooth being surgically cut and restoration being milled since the external surface of the restoration being milled is already known. The dentist or assistant than selects the material for the restoration to be milled based on their clinical assessment which may be present in the user interface. Various options include but are not limited to, composite blocks (inexpensive and fast to mill), lithium disilicate (slow to mill, very strong restoration), porcelain, and the like.

As set forth above, dental treatment system 10 includes at least one tooth scanner 14. In a variation, tooth scanner 14 can operate in several modes. Examples of such modes include, but are not limited to, surgical scan mode where a tooth requiring surgery and two adjacent teeth are imaged, occlusion scan mode in which teeth in an opposing arch of teeth that occlude with the teeth undergoing surgery, interdigitating scan in which a buccal/side scan mode with upper and lower teeth biting together are imaged to register how they fit together.

In certain variations, at least two scans of the subject's teeth will be performed. A first scan is performed before placement of the modified rubber dam and a second scan after placement of the modified rubber dam. With respect to the first scan, the user (e.g., a dental assistant) takes a 3D scan which acts a location map for our software. In a refinement, the user takes a scan of an area recommended by the control program 20. Regions recommended to scan depend on which teeth are being surgically treated. Typically, the scan can be a combination of a surgical scan, an occlusion scan, and/or an interdigitating scan. In a surgical scan, the tooth in question and the two teeth adjacent to it are scanned. In an occlusion scan, the teeth in the opposing arch of teeth that occlude (fit together with) with the teeth undergoing surgery are scanned. In an interdigitating scan, a buccal/side scan with the upper and lower teeth above biting together to register how they fit together is performed. The interdigitating scan image is used to register the position of how the upper and lower teeth scans fit together. With respect to the second scan, the user performs a second 3D scan with the rubber dam, rubber dam frame, modified rubber dam clamp, and passive positioning arm in place. Control program 20 registers or aligns the position of the two scans to create a combined 3D mesh. The combined 3D mesh images a tooth above the rubber dam, the tooth and gum tissue below the rubber dam, and the position of the modified rubber dam clamp positioning reference point.

Control program 20 is advantageously able to direct segmented dental handpiece 16 to perform surgery on multiple teeth while being held by an operator (e.g., a dentist). In particular, dental treatment system 10 can perform quadrant surgery. If the teeth are posterior teeth (from back molar to middle central incisor—International tooth number 1 to 8), scans are completed from posterior molars to the midline. This is called a quadrant in dentistry. Dental treatment system 10 is also able to perform anterior surgery. In particular, if the teeth are anterior teeth (tooth numbers 3-3), quadrants are not used. Instead, a grouping of anterior teeth is used of both upper and lower arches. For example, surgery on a tooth would require a scan of the tooth in question and the two adjacent teeth as the surgical area the opposing teeth. This surgery would also require a scan with the patient biting their teeth together from the side of approximately 15+45, 16+46, 17+47 to register how the teeth fit together. The scan process consists of the user (e.g., assistant) taking multiple pictures which include Z-value depth information which are stitched together to create a 3D model.

These scans can then be used to create a rotatable composite three-dimensional model which is presented to the user. In this model, the user is able to set a preparation zone depending on the preparations type (e.g., crowns/onlays/inlays). In a refinement, the user can digitally paint different areas of the teeth with different “brushes” that signify the depth of cuts under each paint color. In another refinement, the user interacts with the 3d mesh using a haptic stylus input such as: the Geomagic Sculpt (www.geomagic.com/en/products/sculpt/touch/). The orientation of the cuts is perpendicular to the surface of the mesh which is painted or perpendicular to the occlusal plane. In a refinement, a preferred default value for the depth of the cuts is set as the default. In another variation “no-go” zones are set by the dentist and/or the control program 20. For example, 2D planes are set which go between the teeth and act as interproximal barriers to identify where one tooth starts and the next ends. This prevents the control software from extending cuts out of one tooth into another creating the working envelope. In this regard, the dentist sets gingival/gum margins by painting margins with a brush. In a variation, control program 20 is operable to determine the approximate location of a tooth's pulp and/or nerves. In one refinement, the determination is a probabilistic location based on radial distance from outer surface of tooth and likely depth to encounter the nerve.

In some variations, control program 20 requires an indication from the dentist to enable auto-extension of a dental preparation if decay is found during the surgical procedure. When decay is found, the user is presented with several options. The decay can be removed based on criteria set by the dentist. The decay can be left allowing the dentist to program an additional cycle of tooth cutting at this point in the program. The starting point of this cutting step is after the most recent tooth cut. This essentially re-runs the dentist input step using 3D models updated with the tooth status after the first pass of cutting. Problematic decayed areas can be highlighted in a different color on the 3D mesh. Details of when to remove decay can be given to the software. For example, including settings to extend the preparation if it is close to what the dentist or software define as the space likely to contain the nerve. The software could be instructed to chase the decay into that space, or not to at the dentist's discretion. When a treatment plan has been finalized, the user provides an indication in the user interface that the treatment plan is accepted (i.e., checking a box, clicking a button, etc.)

After the user has completed editing of the three-dimensional tool path, the central processing unit is operable to control the segmented dental handpiece to mill internal surface and margins of a target tooth in accordance to regions identified by the user in the three-dimensional model. For example, the segmented dental handpiece 16 is operable to cut out an entire outline form shape as depicted by the user using digital brushes in the input device prior to starting the surgery. In this regard, the central processing unit 12 is operable to delay milling until a tooth preparation shape is completed.

The capture software of the scanner into the system software to allow access to the STL file generated. In a refinement, a dental technician takes a 3D scan to provide a location map. In particular, the technician takes a scan of the area recommended by the software.

In a refinement, the central processing unit 12 is operable to create a three-dimensional density matrix of a subject's tooth to be treated by combining data received from the tooth imaging scanner with data received from the dental drill and/or the tooth scanner Specifically, the load of the motor driving the spindle will increase while cutting higher dentistry materials such as enamel, and decrease while cutting lower density materials such as caries. By combining the motor load (and indirectly spindle speed) with the feed rate of the tool, the 3d surface mesh of the tooth and the 3d coordinates of the end effector, we are able to calculate a relative density for each area being cut. In particular, central processing unit 12 is operable to identify volume units in the three-dimensional density matrix and associate each volume unit associated with an identifier that characterizes a state of the tooth. Examples of such identifiers indicate if a region in the tooth includes hard enamel, decayed enamel, hard dentin, decayed dentin, solid cementum, decayed cementum, and pulp/nerve or a restorative material such as amalgam, composite, gold, and the like. Moreover, identifier may also be used to indicate previous filling (e.g., amalgam, composite, etc.) and previous crowns. Central processing unit 12 performs a predetermined treatment protocol on the volume unit depending on the identifier. The predetermined treatment protocols calculate the relative density of the tooth being cut by taking the milling speed, spindle speed of a burr in the end effector (or increase in current flow to the motor driving the spindle), and rate for moving the segmented dental handpiece. In this regard, central processing unit 12 is operable to direct the end effector of the segmented dental handpiece to cut a subject's the tooth in accordance to regions identified in the three-dimensional density matrix.

In a variation, dental treatment system 10 can also include a density scanner. Referring to FIG. 1, dental treatment system 10 further includes a density scanner 150 that creates a voxel based image (like a CT reconstruction). In such voxel images, the density of teeth is represented by colors. Moreover, the voxel image can be superimposed on the mesh. This display process is similar to CT images which use voxels instead of mesh to display density (radiopacity). However, in this variation, instead of using radiation opacity like CT, the density is determined as resistance to the cutting tool spinning. By combining the spindle speed of the end effector/electrical load on the motor, the known contact surface area of the end effector in contact with the mesh, the movement speed of the mesh relative to the tooth (taking into consideration relativistic speeds of the tooth encoded by the encoder arm), a value is calculated that is a relative density value. This value is compared to laboratory data from known density of test materials.

As the segmented dental handpiece cuts a tooth, a 3D matrix of density values is created and indexed to the XYZ coordinate of the end effector on the segmented dental handpiece. The density value for each XYZ coordinate is calculated by using feed rate of the end effector relative to the patient's tooth and the surface area of the end effector burr in contact with the tooth. Since the 3D mesh of the tooth, the localized position of the burr relative to the tooth mesh, the shape of the burr, the burr's wear rate, and the spindle speed are known, the density value can be determined by correlation to a motor load. A tooth state matrix is built at the same XYZ coordinates. These states are calculated via an algorithm that combines the density values with their radial distance from the external surface of the tooth. This calculation takes advantage of the fact that teeth are composed of various materials layered in somewhat predictable positions. The position of the density value means different things in different areas of the tooth. For example, an area discovered to have a low-density spot relative to the adjacent areas could be a soft enamel zone, or the transition from hard enamel to a hard dentin zone depending on the radial position from the surface of the tooth. This difference is the difference between extending the preparation and maintaining it.

The decision to extend the tool path cycles through each marginal point on the tooth-restoration interface is determined by the state of tooth decay and the location of the coordinate in question (e.g., with the tool through the pre-defined no-go zones defined above (adjacent tooth, gums, pulp)). This cycle repeats until no additional decayed areas are detected, a boundary has been reached, or a call dentist item has been triggered.

With reference to FIGS. 6, 7, and 8, schematics of a user interface 160 displayed on a computer screen for the system of FIG. 1 are provided. FIG. 6 provides a screen shot used for tooth selection 172. Two views are provided in tooth selection 172—a three-dimensional view 174 and a topographical layout view 176. A tooth 158 may be selected from this view. FIG. 7 provides a screen shot for overlay of the bitewing x-ray 168 onto the 3D scanned pre-surgical mesh 170. Post-surgical mesh 172 and restoration mesh 174 are also depicted. The restoration mesh 174 will include the surface defined by pre-surgical mesh 170 and the restoration that is to replace the missing tooth section 176. Moreover, restoration mesh 174 has an inner surface 177 that aligns to the postsurgical mesh 172 and an outer surface 178 that represents the desired restoration. FIG. 8 illustrates generation of the post-surgical mesh pre-op 180. The post-surgical mesh is used to create the toolpath. The post-surgical mesh is generated parametrically. In particular, margin placement 182 is determined by 1 (symmetrical margin placement) or 2 (non-symmetrical margin placement) clicks. The user clicks the mouse first (first, click) to indicate the coronal-apical height position on the exterior surface of the tooth 188 to place a surgical margin on the mesial. The superimposition on the bitewing x-ray 168 allows the user to ensure the margin in below any existing decay or previously existing restorative materials. The second mouse click determines the same data as point B for the distal margin. (note: margin placement 182 may be determined via an alternative process 0 this is one embodiment). The user is presented with a snap showing a range of acceptable locations on the exterior surface of the tooth 158 in which to place the margin. The software alerts the user if they select a margin position that encroaches on the biological width, or alternatively is above the contact point. These two boundaries serve as the snap range for the margin placement 182. The user then inputs the margin width 184 with one click—moving the mouse towards the center of the tooth to be treated in from the external margin placement 182. The user is then presented with a snap that allows the user to accept the ideal axial material reduction thickness. Alternatively, the user may disregard the snap and place the margin deeper into tooth tissue to capture decay or existing restorative material. Next, the user selects the taper of the tooth and the minimum reduction thickness 186 with the final click. Each material has minimum thickness requirements (i.e., reduction thickness). This thickness is achieved by articulating the upper and lower teeth meshes and subtracting the minimum thickness requirements from the height of the opposing tooth into the tooth that will be cut. This is done instead of just removing the minimum thickness from the tooth to be cut because certain parts of the tooth to be cut may be not in contact with the opposing tooth, meaning the gap space can be filled with the restorative material. This results in less tooth structure being removed. In the case of a severely worn surgical tooth, the superimposition of the bitewing x-ray 168 may indicate the post-treatment mesh extending on top of the pulp as shown in the anatomical image (bitewing radiograph). In this case, the clinical and inform the patient of the potential need for a root canal or alternatively complete the root canal prophylactically. The user is presented with a snap that indicated the software calculated ideal taper and reduction thickness 186. The user may accept this snap or change it to her/his desired locations.

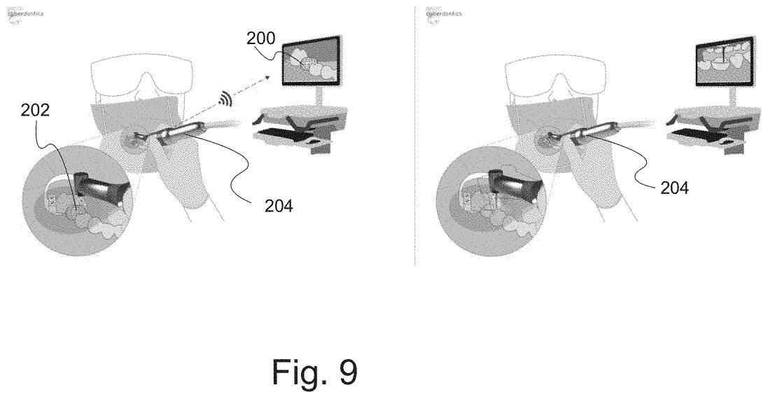

In another embodiment, an alternate usage of the system set forth above when combined with a milling chamber for milling crowns (CAD/CAM system) is provided. Alternatively, any other dental cutting system can be used in this embodiment. With reference to FIG. 9, in this alternative treatment sequence, the patient is diagnosed with a dental problem requiring a dental crown as the solution. This appointment is called the diagnostic appointment and always occurs before the surgical appointment. In this new sequence, the patient's dental arches are scanned at this diagnostic appointment to create a 3D mesh of the surgical area and opposing dental arch. Any additional diagnostic modalities are obtained and registered with the mesh. For example, any color images mapped to the mesh, or radiographs registered to the mesh to help the dentist diagnose and plan the surgery in advance of the surgical procedure.

Between the diagnostic appointment and the surgery appointment, the dentist performs a virtual surgery planning details like the surgical margin location, taper, occlusal reduction, path of insertion, etc. Like pre-planning surgery in the mouth, the dentist will know there are some areas that he may have a degree of uncertainty about. For example, the depth of cares. Interproximal margin placement location can be predictably placed using the registered bitewing x-ray. In addition, an AI comprised of a neural network will compare the crown data to a large data set of previously completed crown surgeries pooled from the aggregate of smartDrills in an anonymized fashion complying with all local legal requirements. With this data, the system can also offer a prediction to the dentist of where the AI believes changes to the margin placement occur. Any changes of internal structure not involving margin placement can be resolved via traditional dental approaches. These include, removing the decay, performing a root canal etc., placing a core, and then having the system mill the internal mating surface of the tooth-crown interface to the pre-planned shape, or having additional reductions filled with cement. Changes in the margin placement have a larger impact on the crown as an open margin is un favorable for high quality dentistry. In these cases, a pre-milled crown will be partially completed. Most structures of the tooth will regardless be predictable. For example, the occlusion, contacts etc. may be predictable and only the margin placement may be uncertain at a specific location. In this case the dentist will approve pre-milling of the majority of the crown, and excess material will be left unmilled at the uncertain location. This partially pre-milled crown can be completed in short time at the surgical appointment once the uncertainly is resolved in much less time that fully milling the crown, as the crown is mostly milled. This allows for shorter time milling at the surgical appointment. Alternatively, no uncertainty exists, allowing the dentist to fully pre-mill the custom crown. Having the custom crown pre-milled is possible because we know the shape the SmartDrill will predictably cut into the tooth. based on the virtual surgery, and the flawless execution of that surgery by the smart drill.

FIG. 9 provides a schematic illustration for making pre-mill crown. As part of preop, a 3d intraoral scan 200 is taken. Radiographs are superimposed on a mesh 202 as set forth above. The crown is then formed using the automated systems and/or segmented drill set 204 forth above. Artificial intelligence can be used to fully or partially pre-mill the crown. On the appointment day, the segmented drill can then be used to prepare the patents tooth for insertion of the crown which is subsequently cemented in place.

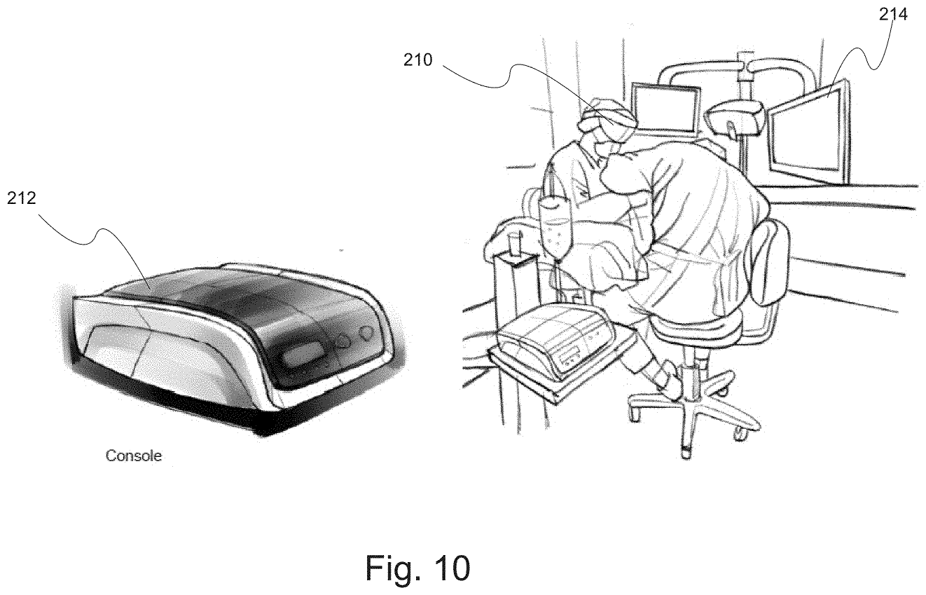

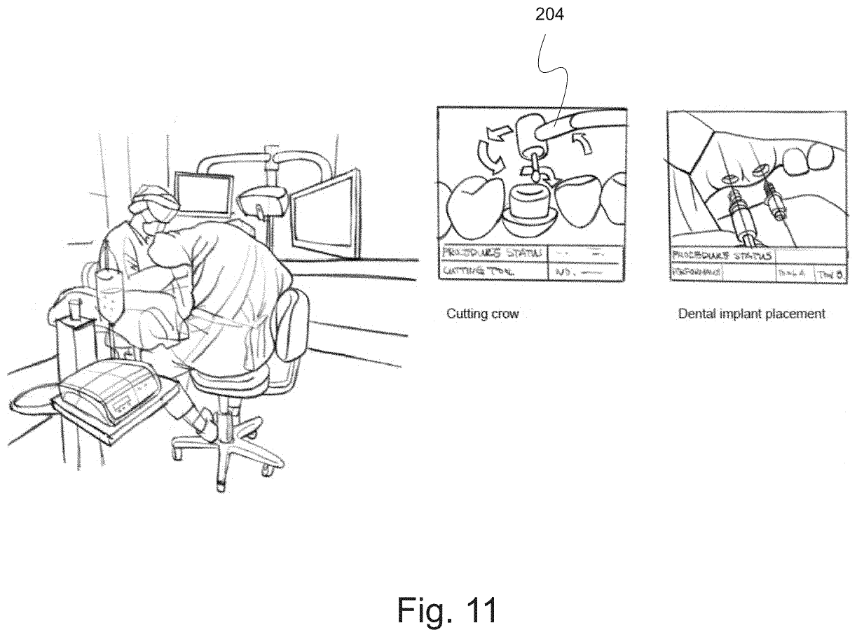

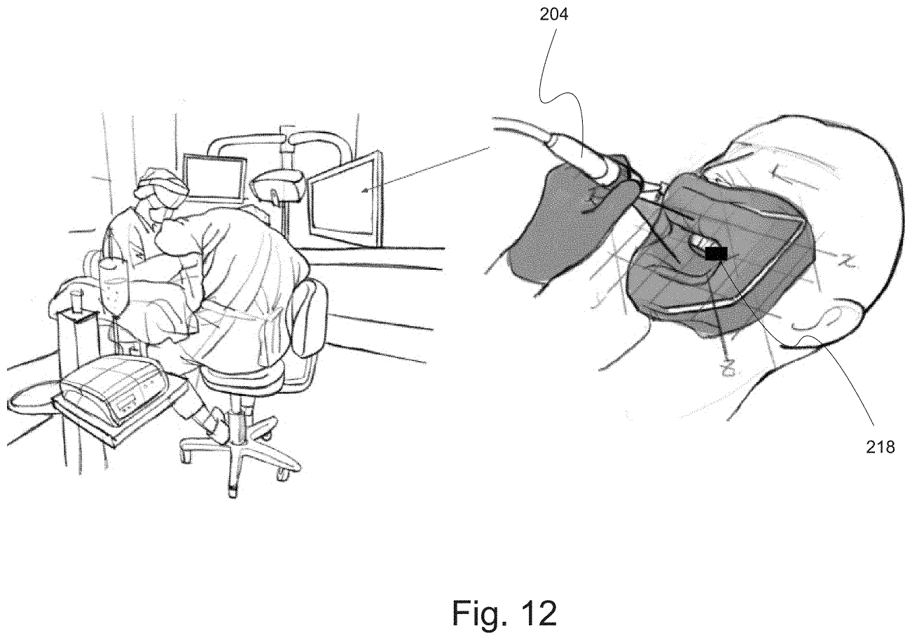

FIGS. 10-13 provides schematics for the use of an augmented reality system in dental applications. In another embodiment, an augmented reality (AR) interface for use with dental instruments includes a user wearable display 210 that is worn by a user which interacts with AR console 212. The system also includes computer/display 214 that provides the display graphics to user wearable display 190. The augmented reality system can be used with traditional dental drills and automated dental systems such as those set forth herein. Moreover, the augmented reality interface can be used with the segmented drill set forth above. As used herein, “augmented reality (AR) refers to a live direct or indirect view of a physical, real-world environment with computer generated augmentations. The augmented reality display superimposes desired tool position, procedure status (if applicable), performance data (if applicable), system information (if applicable), and once the tool is in place the AR displace guides the user to move the tool during the procedure as necessary with images and text over the user's field of view. (see FIG. 11). The user will be able to disable/enable and configure the visible information viewable in the display. The information will be displayed in real time. The display will register the position and movement of the patient using fiducial markers embedded in the dental apparatus installed in the patient's mouth. As depicted in FIG. 12, the display will register the position and movement of the dental equipment using embedded fiducial markers 218.

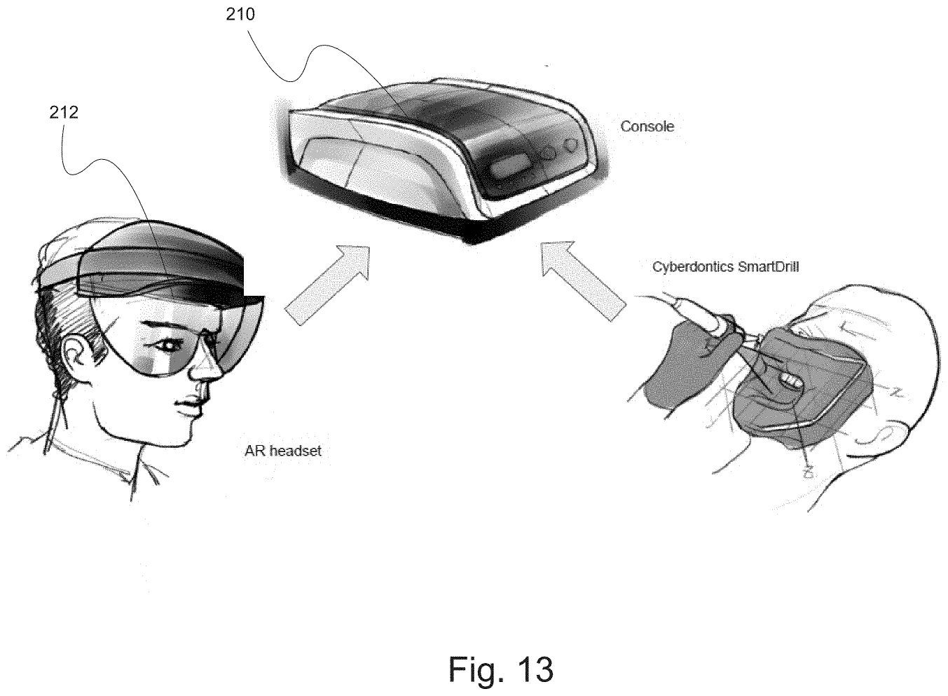

In a variation, the systems set forth above includes manual drills that can be used for dental implant placement, cutting crowns, and any procedure that uses a dental drill. This use also includes automated dental drills like the SmartDrill segment drill set forth herein in FIGS. 1-4. (see, FIG. 13). With SmartDrill systems, the imaging systems tie together positioning data gathered with the smart drill vision systems and integrates it with the data from the AR headset and other positioning systems to create the most complete 3d data set ever.

In another embodiment, a method of performing a dental procedure (e.g., crown restoration) is provided. The method includes forming an external (to a patient) rendering (i.e., a three-dimensional model) of a tooth as is to appear after cutting (e.g., with segmented dental handpiece 16). This three-dimensional rendering can be performed by 3D printing. A crown, bridge, or other detail appliance is then formed external to the patient. segmented dental handpiece 16 or other dental system can be used for this purpose. The crown, bridge, or other detail appliance is then placed within the patient.

With reference to FIG. 1, projection system 230 (e.g., a microlaser projector) can be used to form a laser projection on a tooth combined with a positioning system 232 (e.g., vision, encoder, fixed to the tooth) that provide knowledge of the drills position in 3d space to, a dynamically project a laser beam is fixed on target even as the device moves. The positioning system 230 can be attached to or separate from segmented drill 16. This allows the clinician to see the target location to keep the handpiece on to facilitate maximum device range of motion. The positioning system would compute the location of the tool, and the laser projector would project the target the dental tool user would move the system to. For example, if used with a dental drill, the system would project a laser target onto the tooth, for an implant the laser would project the target onto the gums and bone. The target would be where the user would move the tool to. This solves the problem of having to look at a computer screen or using an AR headset to correctly position the tool.

While exemplary embodiments are described above, it is not intended that these embodiments describe all possible forms of the invention. Rather, the words used in the specification are words of description rather than limitation, and it is understood that various changes may be made without departing from the spirit and scope of the invention. Additionally, the features of various implementing embodiments may be combined to form further embodiments of the invention.

What is claimed is:

1. A system for performing dental surgery on a subject, the system comprising:
a central processing unit that controls automated operation of the system;
a display that renders an image of a target tooth requiring surgical intervention, the image of a target tooth being created from an image file received from the central processing unit;
an input device that receives surgical instructions from a user for providing the surgical intervention, the surgical instructions being received by the central processing unit, the surgical instructions including visual indications on the image of a target tooth that are to be treated;
a segmented dental handpiece having a first segment attached to a second segment, the second segment attached to a dental drill head and being movable with respect to the first segment under control of the central processing unit, the first segment being held stationary relative to the target tooth by an operator, the dental drill head including a dental burr.

2. The system of claim 1 further comprising a three-dimensional (3D) vision system including a plurality of cameras attached to the segmented dental handpiece, the plurality of cameras providing two dimensional images and/or live video of a subject's teeth to be mapped to a predetermined 3D surface scan of a surgical site thereby establishing a world coordinate system to which the segmented dental handpiece is registered.

3. The system of claim 2 wherein the plurality of cameras includes millimeter scale cameras.

4. The system of claim 1 wherein the vision system is adapted to use fiducial markers to located itself in the world coordinate system.

5. The system of claim 4 wherein the fiducial markers include neighboring teeth, optical markers attached to teeth, or dental claps or similar devices.

6. The system of claim 4 wherein the fiducial markers include active emitters to enhance or improve ability of the plurality of cameras to detect or monitor the fiducial markers.

7. The system of claim 1 wherein the segmented dental handpiece includes a micro actuation system that moves the second segment relative to the first segment, the second segment being movable relative to the first segment in 3 to 6 degrees of freedom.

8. The system of claim 7 wherein the segmented dental handpiece includes a motor and a drive shaft that is rotated by the motor, the drive shaft being positionable by the micro actuation system.

9. The system of claim 7 wherein the micro actuation system includes a hexapod micro actuator.

10. The system of claim 7 wherein the micro actuation system includes piezoelectric microactuators.

11. The system of claim 1 wherein the second segment includes a flexible neck.

12. The system of claim 1 wherein the vision system supports a six degree of freedom.

13. The system of claim 1 wherein a boundary for maneuvers of the second segment is defined at least in part by the vision system.

14. The system of claim 1 wherein an output of the vision system is presented in a three-dimensional format either as an overlay onto an imaged anatomy or as a secondary image.

15. The system of claim 1 wherein the central processing unit parametrically generates a 3D mesh of a post-surgical site, and a surgical tool path to be cut into the target tooth.

16. The system of claim 1 wherein the central processing unit executes an algorithm for registering an anatomical image onto a 3d mesh of the surgical site.

17. The system of claim 16 wherein the anatomical image is a radiograph.

18. The system of claim 16 wherein the anatomical image, a registered anatomical image, a 3d representation of the surgical site, and a 3d representation of the target tooth as it will appear post surgically are presented on the display to aid the operator in altering or approving a toolpath generated prior to initiating surgery.

19. The system of claim 1 wherein the system provides operator feedback that guides the operator in positioning of the segmented dental handpiece correctly over the surgical site.

20. The system of claim 19 wherein the operator feedback is tactile or auditory.

21. The system of claim 19 wherein a surgical operation is unlocked once the segmented hand piece is in a correct position.

22. The system of claim 1 wherein the central processing unit monitors cutting performance.

23. The system of claim 1 wherein the central processing unit provides feedback from a burr that is used to monitor burr contact indicating unplanned cutting or to detect tooth decay.

24. The system of claim 1 further comprising a cooling water jet that is used for position tracking, the water cooling jet providing an ultrasound signal or a light signal along a fiber optic axially down the cooling water jet to calculate distance to the target tooth.

25. The system of claim 1 further comprising an external vision system in which camera(s) are external to the segmented dental handpiece.

26. The system of claim 1 wherein the segmented dental handpiece is used without a vison system.

27. The system of claim 26 wherein the segmented dental handpiece is directly fixed to the tooth.

28. The system of claim 1 wherein the segmented dental handpiece includes active emitters and detectors, the emitters emitting light or energy/magnetic waves that are detected by the detectors to determine location of the segmented dental handpiece.

29. The system of claim 1 further comprising guide splits that hold the segmented dental handpiece in certain orientations to cut teeth to fit crowns.

30. A method comprising:
providing the system of any of claims 1 to 29 or another dental cutting system; and
pre-milling of the dental crown prior to a surgical appointment wherein CAD/CAM milling technology is combined with the segmented dental handpiece (i.e., a smart drill) and dental surgery is pre-planned between the diagnostic and surgical appointment based on custom 3D mesh data merged with other diagnostic modalities since the system has programmed therein the shape it is cutting into the tooth before it cuts it, and the segmented dental handpiece can execute the pre-planned surgery with sub millimeter precision cutting the desired shape into the tooth with the milling system milling a crown insert with a mating tooth to crown interface surface before the surgery appointment thereby allowing the crown to be ready chairside at the surgery appointment such that once the surgery is complete the crown is immediately inserted.

31. A method of performing a dental procedure comprises:
forming an external (to a patient) rendering (i.e., a three-dimensional model) of a tooth as is to appear after cutting;
forming a crown, bridge, or other detail appliance external to the patient; and
placing the crown, bridge, or other detail appliance in the patient.

32. The method of claim 31 being performed by the system of claims 1-29.

33. The method of claim 31 wherein the external rendering is formed by 3D printing.

34. The system of any of claim 1-29 further comprising an augmented reality system.

35. The system of claim 34 wherein the augmented reality system includes a user wearable display.

36. The system of claim 34 wherein the augmented reality system further includes a computer that provides the display graphics to user wearable display.

37. The system of any of claims 1-29 wherein the segmented drill manipulated by a user to make broad cuts while the segmented drill automatically makes finer cuts.

38. The system of claims 1-29 further comprising a projection system that can be used to form a laser projection on a tooth and a positioning system that provides knowledge of the segmented dental handpiece's position in 3d space to, a dynamically project a laser beam is fixed on target even as the device moves.
